(19) United States
(12) Patent Application Publication
(10) Pub. No.: US 2008/0097600 A1
(43) Pub. Date: Apr. 24, 2008

(54) MOVABLE OPHTHALMIC LENS ASSEMBLY

(76) Inventor: Ivan Hare, Raanana (IL)

Correspondence Address: David Klein;DEKEL PATENT LTD., Beit HaRof'im, 18 Menuha VeNahala Street, Room 27, REHOVOT, omitted

(21) Appl. No.: 11/552,221

(22) Filed: Oct. 24, 2006

(51) Int. Cl. A61F 2/16; G02C 7/04

(52) U.S. Cl. 623/6.37; 351/161; 623/6.41; 623/6.32; 623/6.13

(57) ABSTRACT

A movable ophthalmic lens assembly including a carrier placeable on a portion of an eye, and a movable ophthalmic lens with non-zero optic power arranged for movement over a surface of the carrier, wherein the movable ophthalmic lens is responsive to ocular muscular movement so as to move in translatory motion over the surface of the carrier.

### FIELD OF THE INVENTION

The present invention relates generally to ophthalmic lenses, such as but not limited to, contact lenses and intraocular lenses, and particularly to movable ophthalmic lenses.

### BACKGROUND OF THE INVENTION

It is well known that lenses have been developed to address the problems encountered by users who require correction of their vision for both near and distance viewing. For example, bifocal and multi-focal contact lenses are well known.

One known bifocal contact lens is known as alternating image bifocals or alternating vision bifocals, which have a distance viewing region located in the upper portion of the lens and a near-viewing portion located in the bottom portion of the lens. The two parts of the lens are separated by a line extending across the lens that may be straight or curved. These bifocal contact lenses function by taking a different position in straight ahead gaze and in down gaze. Theoretically, in straight ahead gaze the pupil is covered by the distance zone and in down gaze the contact lens is higher and the pupil is covered by the near zone.

However, this arrangement suffers from certain disadvantages. One disadvantage of this arrangement is that it is difficult to precisely control the movement of the lens so that the pupil is alternately covered by the near and distance position. To achieve this is a skillful process that is time-consuming and not always successful. Failure to achieve optimal pupil coverage has the disadvantage that the user looks through both the distance and near positions simultaneously resulting at times in two images at once which is known as “ghosting” and habitually in a decrease in visual acuity compared with that achieved with spectacles.

A second disadvantage is that for an alternating vision bifocal to perform optically it is required to move significantly more than is optimum for comfort and thus the user may experience some discomfort. Further it may be necessary to provide features on the lens to control its rotation. However, these features may increase the thickness and the irregularity of the contact lens which may result in lower comfort than the conventional simple design.

An alternative lens arrangement is known under the generic term of simultaneous image bifocal or multifocal. These contact lenses are typically formed from two or more concentric zones of alternating distance and near power or a single zone of continuously changing power (progressive). Two options are available. In the first, a center near contact lens is known in which the near power is at the center of the lens. In the second, a center distance contact lens with the opposite arrangement is known. Typically for the progressive design the near portion will be in the center but the opposite arrangement is known.

One benefit of these lenses is that they do not require movement to perform as bifocals. However, they do require excellent centration during both distance and near gaze. This requirement is a disadvantage compared to single vision contact lenses in making fitting more exacting. The principal disadvantage of these designs is due to the fact that in order to function they focus a distance and near image on the retina at all times.

A further disadvantage is that they produce a retinal image of poorer quality than that obtained with single vision contact lenses or spectacles. For example, during distance gaze only part of the pupil is covered by the distance optics which form the in focus image, the rest of the pupil is covered by out of focus intermediate and/or near zones which degrade the quality of the image produced on the retina and consequently decrease visual performance. A still further disadvantage is that the size of pupils varies between patients and more importantly for each patient with different levels of luminance.

### SUMMARY OF THE INVENTION

The present invention seeks to provide a movable ophthalmic lens, which for example may be movable inside or outside a carrier, as is described more in detail hereinbelow.

The ophthalmic lens assembly of the invention may be used, without limitation, as contact lenses, intraocular lenses, implant lenses, inlay lenses, onlay lenses and other ophthalmic refracture devices.

There is thus provided in accordance with an embodiment of the present invention a movable ophthalmic lens assembly including a carrier placeable on a portion of an eye, and a movable ophthalmic lens with non-zero optic power arranged for movement over a surface of the carrier, wherein the movable ophthalmic lens is responsive to ocular muscular movement so as to move in translatory motion over the surface of the carrier.

The movable ophthalmic lens may be movably disposed in a receptacle formed in the carrier. The receptacle may include a hollow chamber formed inside the carrier, wherein the movable ophthalmic lens is free to move in the chamber. Alternatively, the receptacle may include a track formed in the carrier, wherein the movable ophthalmic lens slides along the track. The movable ophthalmic lens may include a peripheral portion slidingly received in the track and an optics portion with non-zero optic power that is not received in the track.

In accordance with an embodiment of the present invention the carrier may have at least one portion with non-zero optic power and/or at least one portion with zero optic power. The carrier may be formed with an aperture over which the movable ophthalmic lens is movable.

In accordance with an embodiment of the present invention the movable ophthalmic lens may include a portion that is pivotally attached to the carrier. The carrier may include abutments arranged to limit travel of the movable ophthalmic lens with respect to the carrier.

In accordance with an embodiment of the present invention the movable ophthalmic lens may be formed by a liquid that at least partially fills the chamber so as to have non-zero optical power, the liquid permitting light rays to pass therethrough.

The carrier may be adapted to transmit forces from the ocular muscular movement to the movable ophthalmic lens.

### BRIEF DESCRIPTION OF THE DRAWINGS

The present invention will be understood and appreciated more fully from the following detailed description, taken in conjunction with the drawings in which:

FIGS. 1 and 2 are simplified pictorial and sectional side-view illustrations, respectively, of a movable ophthalmic lens assembly, constructed and operative in accordance with an embodiment of the present invention;

FIGS. 2A and 2B are simplified side-view illustrations of the movable lens during downward and upward gazes for near-distance and far-distance viewing, respectively;

FIGS. 3, 4 and 5 are simplified sectional side-view illustrations of other embodiments of movable ophthalmic lens assemblies;

FIGS. 6A and 6B are simplified sectional side-view illustrations of a movable ophthalmic lens assembly, constructed and operative in accordance with another embodiment of the present invention, comprising a movable liquid or gel lens;

FIGS. 7, 7A and 7B are simplified pictorial and side-view illustrations, respectively, of a movable ophthalmic lens assembly, constructed and operative in accordance with yet another embodiment of the present invention; and

FIGS. 8 and 9 are simplified sectional side-view illustrations of other embodiments of movable ophthalmic lens assemblies.

### DETAILED DESCRIPTION OF EMBODIMENTS

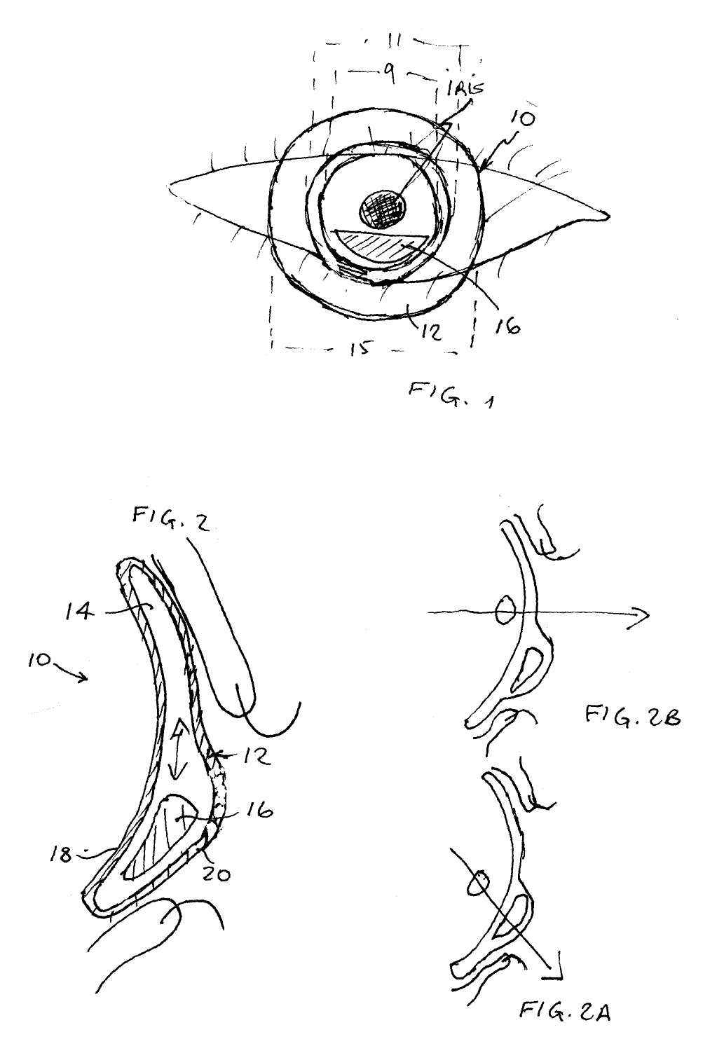

Reference is now made to FIGS. 1 and 2 which illustrate a movable ophthalmic lens assembly 10, constructed and operative in accordance with a preferred embodiment of the present invention.

The movable ophthalmic lens assembly 10 may include a carrier 12 placeable on a portion of an eye. In the illustrated embodiment, the assembly 10 is a contact lens assembly, but it is appreciated that the invention is not limited to this and assembly 10 may be constructed as an IOL or other ocular lens device as well.

Accordingly, in the illustrated embodiment, the carrier 12 may be manufactured from any suitable contact lens material, such as but not limited to, silicone, hydroxyethyl methacrylate (HEMA), or other suitable materials that are biocompatible with the cornea, polymethylmethacrylate (PMMA), silicone rubber, collagen, hydrogel, hyaluronic acid (including the sodium, potassium and other salts thereof), polysulfones, thermolabile materials and other relatively hard or relatively soft and flexible biologically inert materials. Carrier 12 may be formed with a receptacle 14, such as a hollow chamber. A movable ophthalmic lens 16 may be movably disposed in receptacle 14. Lens 16 may be free to move in the hollow chamber. Lens 16 may be manufactured from the same material as carrier 12. Alternatively, lens 16 may be manufactured from a different material than carrier 12.

The outer layers of the carrier 12 may be fused or otherwise joined at their peripheries by any suitable method, such as but not limited to, bonding with adhesive substances, welding/soldering/joining by chemical, laser or any other welding methods, or by making the whole lens with a preformed space using molding or lathe cutting techniques.

The carrier 12 may have no (zero) optical power or may instead have positive or negative optical power for certain needs of the patient, such as to compensate for distance refractive errors. As another example, the inwardly facing surface of the carrier 12 (i.e., the posterior face 18) may have non-zero optical power whereas the outwardly facing surface of the carrier 12 (i.e., the anterior face 20) may have neutral (zero) optical power, or vice versa. Carrier 12 may have a stabilizing shape (e.g., formed by known stabilization techniques such as prism truncation or other stabilization techniques that aid in correct lens location in the eye.

The insert lens 16 may be finely polished to be slippery inside the receptacle 14. The lens 16 is designed to move upwards with the pressure of the lower eye lid upon downward gaze, focusing the light rays for near-distance viewing, e.g., for reading (FIG. 2A). The insert lens 16 moves back down with the pressure of the upper eye lid for far distance vision (FIG. 2B). In one non-limiting embodiment, the lens 16 may have a triangular shape being thicker at its base and thinner at its apex, the base being situated generally parallel to the lower lid. By moving upwards, the insert lens 16 is located in the line of sight of the pupil, creating a refractive change different than the refractive state of the contact lens in the primary position, being straight ahead gaze. Other embodiments will include different shapes and or sizes that fit inside its space of confinement.

The movable ophthalmic lens 16 is thus arranged for movement over a surface of the carrier 12 (e.g., the inner surface thereof), wherein lens 16 is responsive to ocular muscular movement so as to move in translatory motion over the surface of carrier 12.

With the system of the invention, any ametropia may be corrected, in addition to age (or non age) related presbyopia. In addition, emmetropic presbyopia patients would be able to wear the device and see far and near with a functional degree of accuracy.

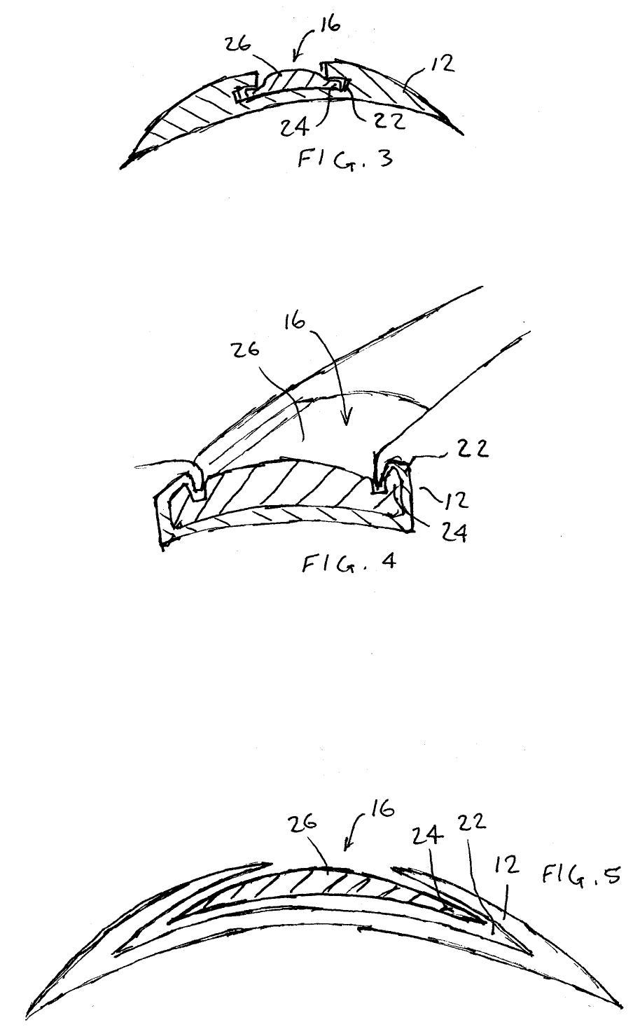

Reference is now made to FIGS. 3, 4 and 5, which illustrate other embodiments of movable ophthalmic lens assemblies. In these embodiments, the movable ophthalmic lens 16 slides along a track 22 formed in carrier 12. The lens 16 may include a peripheral portion 24 slidingly received in track 22, and an optics portion 26 with non-zero optic power (i.e., positive or negative lens) that is not received in track 22.

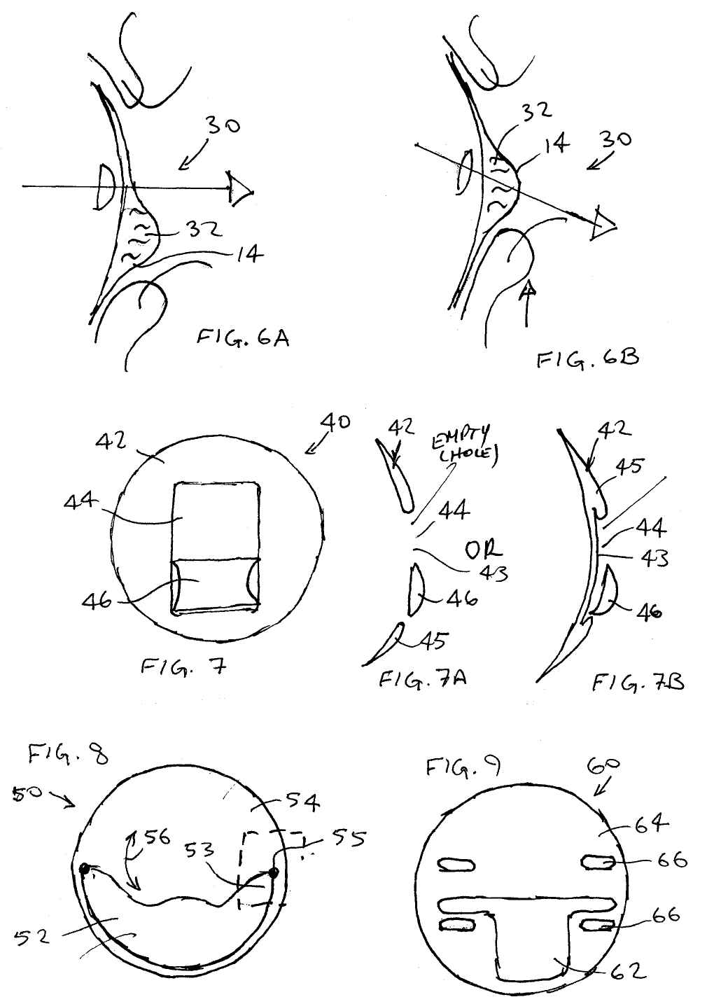

Reference is now made to FIGS. 6A and 6B, which illustrate a movable ophthalmic lens assembly 30, constructed and operative in accordance with another embodiment of the present invention. In this embodiment, the movable ophthalmic lens is formed by a flowable material 32, e.g., a movable liquid or gel (such as water or silicone gel), which at least partially fills the chamber (receptacle) 14 so as to have non-zero optical power. The flowable material 32 is either transparent or translucent, permitting light rays to pass therethrough. The translation of fluid or gel in the sealed pocket or chamber by the action of the eyelids may provide accommodative action and vision correction.

Reference is now made to FIGS. 7, 7A and 7B, which illustrate a movable ophthalmic lens assembly 40, constructed and operative in accordance with yet another embodiment of the present invention. In this embodiment, assembly 40 may include a carrier 42 placeable on a portion of an eye. Carrier 42 may be formed with a receptacle 44, such as a groove or channel, in which a movable ophthalmic lens 46 may be movably disposed. In the version shown in FIG. 7A, receptacle 44 is an aperture over which the movable ophthalmic lens 46 is movable. In the version shown in FIG. 7B, receptacle 44 is a channel. It is seen that the carrier 42 may have at least one portion 43 with zero optic power (such as the aperture in FIG. 7A, or it could be the central portion of the carrier in FIG. 7B in the center of the channel) and/or at least one portion 45 with non-zero optic power.

Reference is now made to FIG. 8, which illustrates another embodiment of a movable ophthalmic lens assembly 50.

In this embodiment, the assembly 50 includes a movable ophthalmic lens 52 which may include a portion 53 that is pivotally attached to a carrier 54 at a pivot 55. In such an arrangement, lens 52 may generally move over the surface of carrier 54 as indicated by arrows 56. The pivoted connection may be a peg or other protruding member that pivots in a hole or slot.

Reference is now made to FIG. 9, which illustrates another embodiment of a movable ophthalmic lens assembly 60.

In this embodiment, the assembly 60 includes a movable ophthalmic lens 62 that moves with respect to a carrier 64 by sliding over an outer or inner surface thereof. The carrier 64 may include abutments 66 arranged to limit travel of the movable ophthalmic lens 62 with respect to the carrier 64. The abutments 66 may also allow directional placement of the lens 62 for orientation proposes.

It is noted that in all of the embodiments, the carrier may transmit forces from the ocular muscular movement to the movable ophthalmic lens.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A movable ophthalmic lens assembly comprising:
a carrier placeable on a portion of an eye; and
a movable ophthalmic lens with non-zero optic power arranged for movement over a surface of said carrier, wherein said movable ophthalmic lens is responsive to ocular muscular movement so as to move in translatory motion over the surface of said carrier.

2. The movable ophthalmic lens according to claim 1, wherein said movable ophthalmic lens is movably disposed in a receptacle formed in said carrier.

3. The movable ophthalmic lens according to claim 2, wherein said receptacle comprises a hollow chamber formed inside said carrier, wherein said movable ophthalmic lens is free to move in said chamber.

4. The movable ophthalmic lens according to claim 2, wherein said receptacle comprises a track formed in said carrier, wherein said movable ophthalmic lens slides along said track.

5. The movable ophthalmic lens according to claim 4, wherein said movable ophthalmic lens comprises a peripheral portion slidingly received in said track and an optics portion with non-zero optic power that is not received in said track.

6. The movable ophthalmic lens according to claim 1, wherein said carrier has at least one portion with non-zero optic power.

7. The movable ophthalmic lens according to claim 1, wherein said carrier has at least one portion with zero optic power.

8. The movable ophthalmic lens according to claim 1, wherein said carrier is formed with an aperture over which said movable ophthalmic lens is movable.

9. The movable ophthalmic lens according to claim 1, wherein said movable ophthalmic lens comprises a portion that is pivotally attached to said carrier.

10. The movable ophthalmic lens according to claim 1, wherein said carrier comprises abutments arranged to limit travel of said movable ophthalmic lens with respect to said carrier.

11. The movable ophthalmic lens according to claim 3, wherein said movable ophthalmic lens is formed by a flowable material that at least partially fills said chamber so as to have non-zero optical power, said flowable material permitting light rays to pass therethrough.

12. The movable ophthalmic lens according to claim 1, wherein said carrier is adapted to transmit forces from the ocular muscular movement to said movable ophthalmic lens.

13. The movable ophthalmic lens according to claim 1, wherein said carrier and said movable ophthalmic lens together comprise a contact lens.

14. The movable ophthalmic lens according to claim 1, wherein said carrier and said movable ophthalmic lens together comprise an intraocular lens.
